(19) United States
(12) Patent Application Publication
(10) Pub. No.: US 2023/0076154 A1
(43) Pub. Date: Mar. 9, 2023

(54) METHOD OF PREDICTION WITH RESPECT TO HEALTH CARE PROVIDERS AND SYSTEM THEREOF

(71) Applicant: VERIX LTD., Herzliya (IL)

(72) Inventors: Elad Haver, Kfar Saba (IL); Liron Grinwald, Netanya (IL); Shahar Yitshak Cohen, Kiryat Ono (IL)

(21) Appl. No.: 17/903,308

(22) Filed: Sep. 6, 2022

Related U.S. Application Data
(60) Provisional application No. 63/242,309, filed on Sep. 9, 2021.

(51) Int. Cl. G16H 40/20; G06Q 10/06

(52) U.S. Cl. CPC G16H 40/20; G06Q 10/06375

(57) ABSTRACT

A prediction method and system. The method includes obtaining a health care provider (HCP) life cycle comprising a plurality of stages indicative of states of an HCP with respect to a medical product, and one or more transitions each between a pair of stages; for a given transition from a first stage to a second stage, obtaining a first list of HCPs associated with the first stage indicative of a present state of the HCPs at a present time point, and first attribute data characterizing the first list of HCPs; and performing a prediction with respect to the transition using a ML model based on the first attribute data, giving rise to a second list of HCPs each associated with a respective likelihood of changing states corresponding to the transition. The ML model is trained with respect to the transition using training data pertaining to a given time period.

### TECHNICAL FIELD

The presently disclosed subject matter relates, in general, to the field of predictive analytics, and more specifically, to machine learning based data prediction.

### BACKGROUND

With rapid development of industrial processes and computerization, it is recognized in today's digital economy that enterprises and organizations must timely monitor their organizational data to support strategic planning and decision making. This is particularly true with respect to medical and pharmaceutical organizations which may possess massive data, thus facing challenges with respect to data management and analysis. These organizations must become data-driven in order to improve performance and remain sustainable and competitive.

Machine learning technology has been recently employed in certain fields to analyze enterprise data and predict likely outcomes, which may benefit organizations by automating the processes, making data-driven decisions, and improving the efficiency and accuracy of organizational operations. However, current machine learning based systems are still limited in their usages with respect to certain applications, due to various limitations.

### SUMMARY

In accordance with certain aspects of the presently disclosed subject matter, there is provided a computerized prediction method, the method performed by a processing and memory circuitry (PMC) and comprising: obtaining a predefined health care provider (HCP) life cycle comprising: a plurality of stages indicative of a plurality of states of an HCP with respect to a given medical product, and one or more transitions each between a pair of stages of the plurality of stages and indicative of a change of state of an HCP between two states corresponding to the pair of stages; for at least one given transition from a first stage to a second stage in the HCP life cycle, obtaining a first list of HCPs associated with the first stage indicative of a present state of the HCPs with respect to the given medical product at a present time point, and first attribute data characterizing the first list of HCPs at the present time point; and performing a prediction on the first list of HCPs with respect to the given transition using a machine learning (ML) model based on the first attribute data, giving rise to a second list of HCPs, each associated with a respective likelihood of changing states corresponding to the given transition at the present time point; wherein the ML model is previously trained with respect to the given transition using training data pertaining to a given time period, the training data including historical attribute data characterizing a historical first list of HCPs associated with the first stage at the beginning of the given time period, and ground truth data indicative of a result of change of state for each HCP in the historical first list at the end of the given time period.

In addition to the above features, the method according to this aspect of the presently disclosed subject matter can comprise one or more of features (i) to (ix) listed below, in any desired combination or permutation which is technically possible:
(i). The plurality of stages comprises at least two of the following stages: not a prescriber, a new prescriber, a continuous prescriber, an increasing prescriber, a decreasing prescriber, and a churned prescriber.
(ii). The ML model is selected from a group comprising: decision tree, Support vector machine (SVM), Artificial neural network (ANN), Bayesian network, and an ensemble thereof.
(iii). The first attribute data comprises one or more attributes from a set of attributes characterizing the HCPs in the first list including: specialty, geography, historical number of patients, historical number of prescriptions, acquisition rate of new patients, tendency to switch between medical products, patient attributes, and historical events directed to the HCPs.
(iv). The given time period includes one or more sub-periods within the given time period, and wherein the ML model is trained using training data from each of the one or more sub-periods.
(v). The at least one given transition comprises a plurality of selected transitions, and the method comprises, for each selected transition, obtaining a respective first list of HCPs associated with a respective first stage at the present time point and respective first attribute data characterizing the respective first list of HCPs, and performing a prediction on the respective first list of HCPs with respect to the selected transition using a respective ML model based on the respective first attribute data at the present time point, giving rise to a plurality of second lists of HCPs corresponding to the plurality of selected transitions at the present time point.
(vi). The plurality of selected transitions are selected from a group comprising: a transition between not a prescriber and a new prescriber, a transition between a new prescriber and a continuous prescriber, a transition between a continuous prescriber and a churned prescriber, a transition between a churned prescriber and a new prescriber, a transition between a continuous prescriber and an increasing prescriber, and a transition between a continuous prescriber to a decreasing prescriber.
(vii). The respective ML model is specifically selected according to the selected transition.
(viii). The given medical product is selected from a group comprising: a given medicine, a given medical service, a given medical device, or a given brand of medicines.
(ix). The second list of HCPs is usable for prioritizing HCPs for an event directed to the given transition.

In accordance with other aspects of the presently disclosed subject matter, there is provided a computerized prediction system, the system comprising a processor and memory circuitry (PMC) configured to: obtain a predefined health care provider (HCP) life cycle comprising: a plurality of stages indicative of a plurality of states of an HCP with respect to a given medical product, and one or more transitions each between a pair of stages of the plurality of stages and indicative of a change of state of an HCP between two states corresponding to the pair of stages; for at least one given transition from a first stage to a second stage in the HCP life cycle, obtain a first list of HCPs associated with the first stage indicative of a present state of the HCPs with respect to the given medical product at a present time point, and first attribute data characterizing the first list of HCPs at the present time point; and perform a prediction on the first list of HCPs with respect to the given transition using a machine learning (ML) model based on the first attribute data, giving rise to a second list of HCPs each associated with a respective likelihood of changing states corresponding to the given transition at the present time point; wherein the ML model is previously trained with respect to the given transition using training data pertaining to a given time period, the training data including historical attribute data characterizing a historical first list of HCPs associated with the first stage at the beginning of the given time period, and ground truth data indicative of a result of change of state for each HCP in the historical first list at the end of the given time period.

This aspect of the disclosed subject matter can comprise one or more of features (i) to (ix) listed above with respect to the method, mutatis mutandis, in any desired combination or permutation which is technically possible.

In accordance with other aspects of the presently disclosed subject matter, there is provided a non-transitory computer readable medium comprising instructions that, when executed by a computer, cause the computer to perform a prediction method, the method comprising: obtaining a predefined health care provider (HCP) life cycle comprising: a plurality of stages indicative of a plurality of states of an HCP with respect to a given medical product, and one or more transitions each between a pair of stages of the plurality of stages and indicative of a change of state of an HCP between two states corresponding to the pair of stages; for at least one given transition from a first stage to a second stage in the HCP life cycle, obtaining a first list of HCPs associated with the first stage indicative of a present state of the HCPs with respect to the given medical product at a present time point, and first attribute data characterizing the first list of HCPs at the present time point; and performing a prediction on the first list of HCPs with respect to the given transition using a machine learning (ML) model based on the first attribute data, giving rise to a second list of HCPs each associated with a respective likelihood of changing states corresponding to the given transition at the present time point; wherein the ML model is previously trained with respect to the given transition using training data pertaining to a given time period, the training data including historical attribute data characterizing a historical first list of HCPs associated with the first stage at the beginning of the given time period, and ground truth data indicative of a result of change of state for each HCP in the historical first list at the end of the given time period.

This aspect of the disclosed subject matter can comprise one or more of features (i) to (ix) listed above with respect to the method, mutatis mutandis, in any desired combination or permutation which is technically possible.

### BRIEF DESCRIPTION OF THE DRAWINGS

In order to understand the disclosure and to see how it may be carried out in practice, embodiments will now be described, by way of non-limiting example only, with reference to the accompanying drawings, in which:

FIG. 1 illustrates a functional block diagram of a prediction system in accordance with certain embodiments of the presently disclosed subject matter.

FIG. 2 illustrates a generalized flowchart of HCP prediction in accordance with certain embodiments of the presently disclosed subject matter.

FIG. 3 illustrates a generalized flowchart of training the ML model in accordance with certain embodiments of the presently disclosed subject matter.

FIG. 4 illustrates a generalized flowchart of performing prediction for a plurality of selected transitions in accordance with certain embodiments of the presently disclosed subject matter.

FIG. 5 illustrates a generalized flowchart of a runtime retraining process of the ML model based on updated training data in accordance with certain embodiments of the presently disclosed subject matter.

FIG. 6 illustrates an exemplary HCP life cycle in accordance with certain embodiments of the presently disclosed subject matter.

### DETAILED DESCRIPTION OF EMBODIMENTS

In the following detailed description, numerous specific details are set forth in order to provide a thorough understanding of the disclosure. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “obtaining”, “performing”, “training”, “selecting”, “predicting”, “prioritizing”, or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including, by way of non-limiting example, the prediction system and respective parts thereof disclosed in the present application.

The terms “non-transitory computer-readable memory” and “non-transitory computer-readable storage medium” used herein should be expansively construed to cover any volatile or non-volatile computer memory suitable to the presently disclosed subject matter. The terms should be taken to include a single medium or multiple media (e.g., a centralized or distributed database, and/or associated caches and servers) that store the one or more sets of instructions. The terms shall also be taken to include any medium that is capable of storing or encoding a set of instructions for execution by the computer and that cause the computer to perform any one or more of the methodologies of the present disclosure. The terms shall accordingly be taken to include, but not be limited to, a read only memory (“ROM”), random access memory (“RAM”), magnetic disk storage media, optical storage media, flash memory devices, etc.

Embodiments of the presently disclosed subject matter are not described with reference to any particular programming language. It will be appreciated that a variety of programming languages may be used to implement the teachings of the presently disclosed subject matter as described herein.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus the appearance of the phrase “one case”, “some cases”, “other cases” or variants thereof does not necessarily refer to the same embodiment(s).

It is appreciated that, unless specifically stated otherwise, certain features of the presently disclosed subject matter, which are described in the context of separate embodiments, can also be provided in combination in a single embodiment.

Conversely, various features of the presently disclosed subject matter, which are described in the context of a single embodiment, can also be provided separately or in any suitable sub-combination. In the following detailed description, numerous specific details are set forth in order to provide a thorough understanding of the methods and apparatus.

In embodiments of the presently disclosed subject matter, one or more stages illustrated in the figures may be executed in a different order and/or one or more groups of stages may be executed simultaneously, and vice versa.

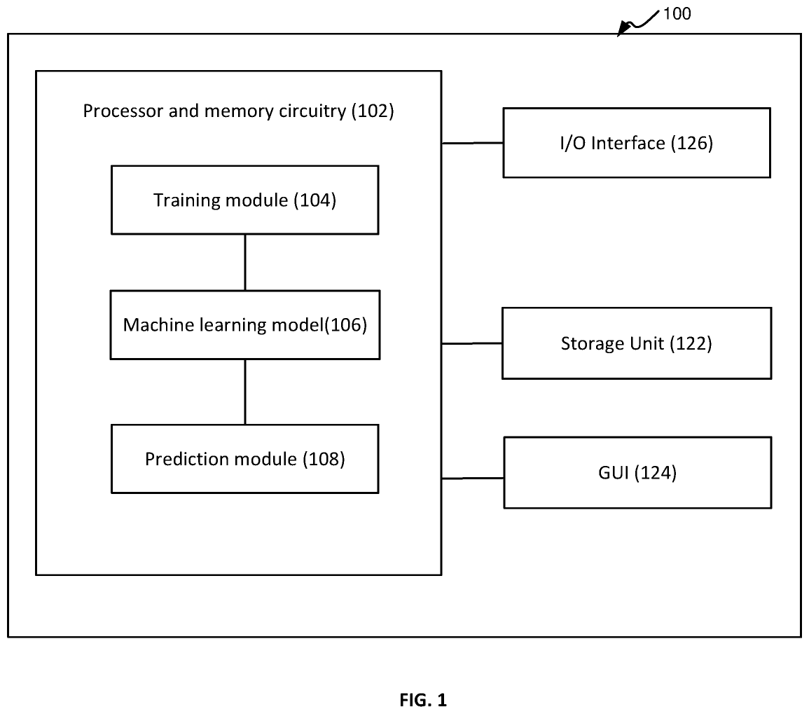

Bearing this in mind, attention is drawn to FIG. 1 illustrating a functional block diagram of a prediction system in accordance with certain embodiments of the presently disclosed subject matter.

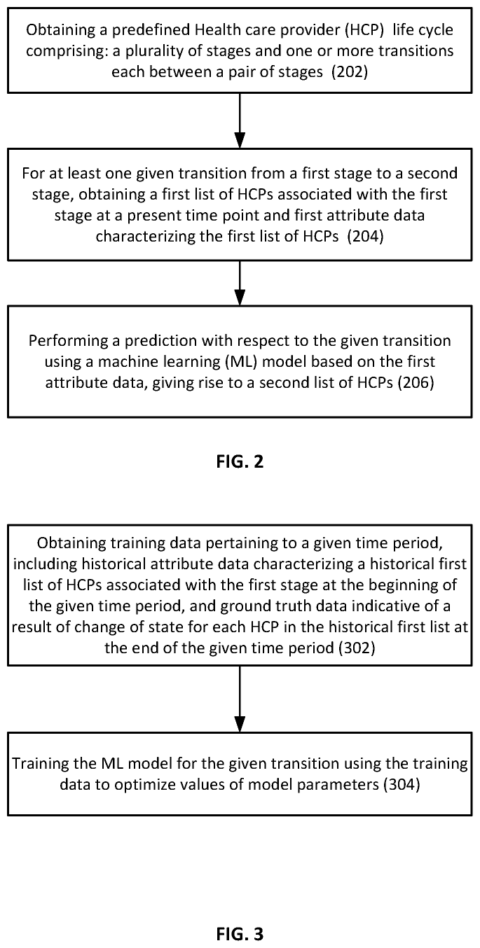

The system 100 illustrated in FIG. 1 is a computer-based system that can be used for performing prediction with respect to an organization, a specific population, and/or a specific field/subject, etc. According to certain embodiments of the presently disclosed subject matter, the system 100 can be configured to perform prediction with respect to health care providers (HCP) in a HCP life cycle based on machine learning technologies, as will be described below in further detail with reference to FIGS. 2-5. System 100 is thus also referred to as a HCP prediction system or a prediction system in the present disclosure.

The term HCP used herein refers to a health care provider that is an individual health professional or a health facility organization licensed to provide health care services including medication, treatments, surgery and medical devices, etc. By way of example, the individual HCPs can refer to any of the following health professionals:

physicians, doctors, specific practice providers, clinicians, and specialists, etc. The institutional HCPs can refer to any of the following health facility organizations: hospitals, clinics, and medical institutes, etc. The present disclosure is not limited to a specific type of HCPs.

In some embodiments, system 100 can be operatively connected to one or more data management systems (not shown in FIG. 1). The term “data management system” referred to herein should be expansively construed to cover any enterprise management system(s) (e.g., enterprise resource planning (ERP), customer relationship management (CRM), etc.) and/or an internal database of such systems which are configured to store and manage raw data and/or structured data related to an organization and the organizational entities thereof

By way of example, in cases where the organization is a medical or pharmaceutical organization, the data management system used thereof can be configured to store and manage organizational data related to medical products as well as data related to operations and customers, etc. In some embodiments, the system 100 can be further operatively connected to external data repositories and/or third party systems for storing and providing necessary data.

Such organizational data, when represented and analyzed properly, can reflect meaningful statistics and characteristics of the data and indicate how certain properties of the data may change over time. Therefore, it may be desirable to have a prediction system that can accurately predict future data related to various organizational aspects based on historical data that have been monitored over time, thereby allowing improved planning and resources allocations. Such an improved planning and resources allocation can in turn increase revenues/sales of the medical products of the organization.

Continuing with the above example where the organization is a medical/pharmaceutical organization, the organizational products can include at least one of the following medical products: one or more medicines (or a brand of medicines), medical services, and medical devices. The customers of such an organization can include the HCPs as described above. For instance, the customers can be the individual HCPs (e.g., physicians, doctors, etc.) that can prescribe the medical products of the organization, such as the medicines and/or medical services thereof, to their patients. In another example, the customers can be institutional HCPs such as clinics that comprise a plurality of health professionals that can prescribe the medical products such as the medicines and/or medical services thereof. In yet a further example, the customers can be institutional HCPs that may obtain/acquire the medical products, such as medical devices, from the organization.

In such cases, the organization may be desired to model and analyze historical data related to such products and customers in order to provide an indication with respect to future events in these aspects. By way of example, the organization may wish to prioritize its customers (e.g., HCPs), e.g., to select HCPs with relatively high likelihood for prescribing the medical products of the enterprise, and plan accordingly tailored activities/events directed to the selected HCPs. Such prioritization is conventionally performed manually based on statistical analysis of the enterprise data as described above, which is cumbersome, time-consuming, and, in some cases, error-prone. In addition, the HCPs are usually at various states of a life cycle with respect to prescribing the medical products of the enterprise, and may transit to different states over time, thus making the prioritization of relevant HCPs even more complicated.

According to certain embodiments of the presently disclosed subject matter, the proposed prediction system is specifically designed and configured to automate the prioritization of the HCPs with respect to their respective states in relation to the medical products and different transitions in the HCP life cycle, which is not only computationally efficient, but also results in more relevant prediction outcomes, with higher accuracy and lower error rates.

Prediction system 100 includes a processor and memory circuitry (PMC) 102 operatively connected to a hardware-based I/O interface 126. PMC 102 is configured to provide all processing necessary for operating the system 100 as further detailed with reference to FIG. 2 and comprises a processor (not shown separately in FIG. 1) and a memory (not shown separately in FIG. 1). The processor of PMC 102 can be configured to execute several functional modules in accordance with computer-readable instructions implemented on a non-transitory computer-readable memory or storage medium comprised in the PMC. Such functional modules are referred to hereinafter as comprised in the PMC.

The processor referred to herein can represent one or more general-purpose processing devices such as a microprocessor, a central processing unit, or the like. More particularly, the processor may be a complex instruction set computing (CISC) microprocessor, a reduced instruction set computing (RISC) microprocessor, a very long instruction word (VLIW) microprocessor, or a processor implementing other instruction sets, or processors implementing a combination of instruction sets. The processor may also be one or more special-purpose processing devices such as an application specific integrated circuit (ASIC), a field programmable gate array (FPGA), a digital signal processor (DSP), a network processor, or the like. The processor is configured to execute instructions for performing the operations and steps discussed herein.

The memory referred to herein can comprise a main memory (e.g., read-only memory (ROM), flash memory, dynamic random access memory (DRAM) such as synchronous DRAM (SDRAM) or Rambus DRAM (RDRAM), etc.), and a static memory (e.g., flash memory, static random access memory (SRAM), etc.).

In certain embodiments, functional modules comprised in PMC 102 can include a training module 104, a machine learning (ML) model 106, a prediction module 108 which are operatively connected therebetween. The PMC 102 can be configured to obtain, via an I/O interface 126, a predefined HCP life cycle comprising: a plurality of stages indicative of a plurality of states of an HCP with respect to a given medical product, and one or more transitions each between a pair of stages of the plurality of stages and indicative of a change of state of an HCP between two states corresponding to the pair of stages.

The PMC 102 can be further configured to obtain, via an I/O interface 126, for at least one given transition from a first stage to a second stage in the HCP life cycle, a first list of HCPs associated with the first stage indicative of a present state of the HCPs with respect to the given medical product at a present time point, and first attribute data characterizing the first list of HCPs at the present time point.

The prediction module 108 can be configured to use the pre-trained ML model 106 to perform HCP prediction in the inference phase (also referred to as prediction phase, runtime phase, etc.). Specifically, for the at least one given transition, the prediction module 108 can be configured to perform a prediction on the first list of HCPs with respect to the given transition using a ML model (e.g., the ML model 106) based on the first attribute data, giving rise to a second list of HCPs each associated with a respective likelihood of changing states corresponding to the given transition at the present time point.

The ML model 106 is previously trained, e.g., by the training module 104, with respect to the given transition, using training data pertaining to a given time period, the training data including historical attribute data characterizing a historical first list of HCPs associated with the first stage at the beginning of the given time period, and ground truth data indicative of a result of change of state for each HCP in the historical first list at the end of the given time period. The values of the model parameters are automatically tuned/optimized during the training phase.

Operation of system 100, PMC 102 and the functional modules therein will be further detailed with reference to FIGS. 2-5.

According to certain embodiments, the ML model 106 referred to herein can be implemented as various types of machine learning models, such as, e.g., decision tree, Support Vector Machine (SVM), Artificial Neural Network (ANN), Bayesian network, or ensembles/combinations thereof etc. In some cases, the ML model can also be implemented using a regression model, such as, e.g., a linear regression model, or a non-linear regression model, etc. The learning algorithm used by the ML model can be any of the following: supervised learning, unsupervised learning, or semi-supervised learning, etc. The presently disclosed subject matter is not limited to the specific type of ML model or the specific type or learning algorithm used by the ML model.

By way of example, the ML model 106 can be implemented using a Random Forest (RF) model, which is an ensemble of multiple decision trees.

In some embodiments, the ML model 106 can be implemented as a Deep Neural Network (DNN) which includes layers organized in accordance with respective DNN architectures. By way of non-limiting example, the layers of DNN can be organized in accordance with Convolutional Neural Network (CNN) architecture, Recurrent Neural Network architecture, Recursive Neural Networks architecture, Generative Adversarial Network (GAN) architecture, or otherwise.

A set of input data used to adjust the model parameters of a ML model is referred to hereinafter as a training set or training data. As aforementioned, training of the ML model can be performed by the training module 104 during the training phase, as will be detailed below with reference to FIG. 3.

It is noted that the above described ML model types are for exemplary purposes to illustrate possible ways of implementing the ML model, and the teachings of the presently disclosed subject matter are not bound by the specific type of ML model and the architecture thereof.

According to certain embodiments, system 100 can comprise a storage unit 122. The storage unit 122 can be configured to store any data necessary for operating system 100, e.g., data related to input and output of system 100, as well as intermediate processing results generated by system 100. By way of example, the storage unit 122 can be configured to store the training data, the trained ML model, the input data to the ML model, and the prediction result, etc. Accordingly, necessary data and/or models can be retrieved from the storage unit 122 and provided to the PMC 102 for further processing. Alternatively, these data can be stored in a different system (e.g., the enterprise management system) or data repository (which may be located either locally or remotely) that are operatively connected to system 100, and can be retrieved by system 100 through an I/O interface 126.

In some embodiments, system 100 can optionally comprise a computer-based graphical user interface (GUI) 124 which is configured to enable user-specified inputs related to system 100. The user may be provided, through the GUI, with options of defining certain operation parameters. For instance, in some cases, the user can be presented with an interface to provide a request of a specific prediction. The user may also view the prediction results through the GUI, such as, e.g., a list of predicted HCPs. In some cases, optionally, the user can provide feedback on the prediction result through the GUI. The prediction result can also be sent, through the I/O interface 126, to a different system (e.g., the enterprise management system) or data repository that are operatively connected to the system 100 for further rendering.

Those versed in the art will readily appreciate that the teachings of the presently disclosed subject matter are not bound by the system illustrated in FIG. 1. Equivalent and/or modified functionality can be consolidated or divided in another manner and can be implemented in any appropriate combination of software with firmware and/or hardware.

It is noted that the system 100 illustrated in FIG. 1 can be implemented in a distributed computing environment, in which the aforementioned functional modules shown in FIG. 1 can be distributed over several local and/or remote devices, and can be linked through a communication network.

By way of example, although the PMC 102 in FIG. 1 comprises both the training module 104 and the prediction module 108, this is not necessarily so. These modules can be located in separate systems at different places/entities. For instance, the training module 104 can reside in a training system located at a first entity, where the ML model is previously trained using the training data, whereas the prediction module 108 can reside in a prediction system (e.g., the present prediction system 100) located at a second entity that is different from the first entity, where the inference phase prediction is performed using the trained ML model.

It is further noted that in some cases, at least part of the ML model 106, storage unit 122 and/or GUI 124 can be external to the system 100 and operate in data communication with system 100 via I/O interface 126. By way of example, the ML model can be pre-trained and stored externally and can be obtained and processed by system 100 via I/O interface 126. Alternatively, the respective functions of the ML model can, at least partly, be integrated with system 100, thereby facilitating and enhancing the functionalities of the system. By way of another example, the data repositories or the storage unit therein can be shared with other systems or be provided by other systems, including third party equipment.

It is noted that the presently disclosed prediction system 100 can be implemented in a computer or a computerized machine within which a set of instructions, for causing the machine to perform any one or more of the methodologies discussed herein, may be executed. In alternative implementations, the machine may be connected (e.g., networked) to other machines in a LAN, an intranet, an extranet, and/or the Internet. The machine may operate in the capacity of a server or a client machine in a client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is described, the term “machine” shall also be taken to include any collection of machines that individually or jointly execute a set (or multiple sets) of instructions to perform any one or more of the methodologies discussed herein.

While not necessarily so, the process of operation of system 100 can correspond to some or all of the stages of the methods described with respect to FIGS. 2-5. Likewise, the methods described with respect to FIGS. 2-5 and their possible implementations can be implemented by system 100. It is therefore noted that embodiments discussed in relation to the methods described with respect to FIGS. 2-5 can also be implemented, mutatis mutandis as various embodiments of the system 100, and vice versa.

Referring to FIG. 2, there is illustrated a generalized flowchart of HCP prediction in accordance with certain embodiments of the presently disclosed subject matter.

A predefined HCP life cycle can be obtained (e.g., by the PMC 102 of system 100). The HCP life cycle can comprise a plurality of stages indicative of a plurality of states of an HCP with respect to a given medical product, and one or more transitions each between a pair of stages of the plurality of stages and indicative of a change of state of an HCP between two states corresponding to the pair of stages.

As described above, in some cases, the HCPs used herein can refer to individual HCPs such as, e.g., physicians, doctors, specific practice providers, clinicians, and specialists, etc. Alternatively, in some other cases, the HCPs can refer to institutional HCPs such as, e.g., hospitals, clinics, and medical institutes, etc. According to certain embodiments, a given medical product of a medical/pharmaceutical organization can be selected from a group comprising: a given medicine, a given medical service, a given medical device, or a given brand of medicines. The HCPs may choose to prescribe the given medical product (or alternative medical products from other medical companies with similar effects) to their patients for purpose of providing medical treatments. Therefore, such HCPs can be regarded as customers (or potential customers) of the medical/pharmaceutical organization.

The HCPs may be at different states/relationships with respect to prescribing the given medical product. By way of example, an HCP may have never prescribed the given medical product, thus is not a prescriber of the product. By way of another example, an HCP may have just started prescribing the product (e.g., for a relatively short time period), thus is rendered as a new prescriber of the produdct. Similarly, an HCP can be regarded as a continuous prescriber of the product if he/she has been prescribing the product for a relatively longer time period. In yet further examples, an HCP can be regarded as an increasing prescriber whose prescriptions of the given product continue to increase for a certain time period, or a decreasing prescriber whose prescriptions of the given product have been decreasing for a certain time period, or a churned prescriber (i.e., a withdrawing prescriber that ceases his/her relationship with the given product) that used to prescribe the given product, but no longer prescribes it at the present state.

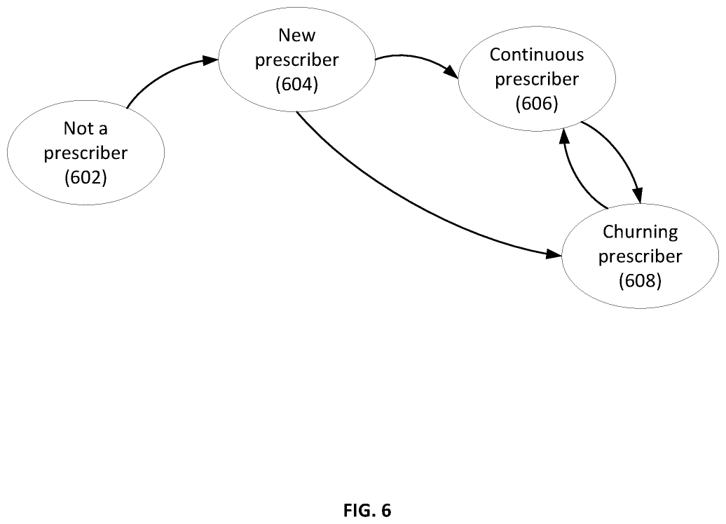

Accordingly, a HCP life cycle can be defined to reflect various states of the HCPs with respect to a given medical product. In addition, over time, the HCPs may undergo various transitions between different states with respect to the given medical product. Therefore, the HCP life cycle also reflects one or more transitions, each between a pair of stages (indicative of two corresponding states). Each transition is indicative of a change of state of an HCP between two corresponding states. Turning now to FIG. 6, there is illustrated an exemplary HCP life cycle in accordance with certain embodiments of the presently disclosed subject matter.

The HCP life cycle as shown in FIG. 6 includes four stages indicative of four corresponding states of an HCP with respect to prescribing a given medical product: a first stage 602—not a prescriber, a second stage 604—a new prescriber, a third stage 606—a continuous prescriber and a fourth stage 608—a churned prescriber. The arrow lines connecting the stages represent the transitions between different stages and the directions of the transitions. Each transition is between a pair of stages among the four stages and is indicative of a change of state of an HCP between the two states corresponding to the pair of stages. For instance, the transitions as illustrated in FIG. 6 include: a transition between not being a prescriber and a new prescriber (i.e., the HCP changes states from not being a prescriber of the product to a new prescriber of the product), a transition between a new prescriber and a continuous prescriber (i.e., the HCP changes states from a new prescriber of the product to a continuous prescriber of the product), two-way transitions between a continuous prescriber and a churned prescriber (i.e., the HCP changes states from a continuous prescriber of the product to not prescribing the product, thus leaving the product/brand, or the other way around), and a transition between a new prescriber and a churned prescriber (i.e., the HCP changes states from a new prescriber of the product to no longer prescribing the product, thus ceasing the relationship with the product/brand).

It is to be noted the above-described stages/states and transitions are illustrated for exemplary purposes only, and should not be deemed as limiting the present disclosure in any way. In some cases, the HCP life cycle can include one or more different stages/states in addition to or in lieu of the above exemplary ones, while in some other cases, the HCP life cycle can include less stages/states. For instance, the HCP life cycle can include additional stages such as, e.g., an increasing prescriber and/or a decreasing prescriber. Similarly, in some cases, there can be additional or alternative transitions (and/or transitions with different directions) between the stages in the life cycle. As described above, the HCPs in the examples can be individual HCPs or institutional HCPs, and the given product may vary, pertaining to different scenarios.

According to certain embodiments, a prediction can be performed for at least one given transition in the HCP life cycle, i.e., to predict the likelihood of the HCPs undergoing such transition at a given time point. In some cases, the at least one given transition can be predetermined, or alternatively can be selected from the plurality of transitions in the life cycle.

For performing the prediction directed to a given transition from a first stage to a second stage at a present time point, a first list of HCPs associated with the first stage at the present time point, and first attribute data characterizing the first list of HCPs at the present time point, can be obtained (204) (e.g., by the PMC 102 of system 100). The first stage is indicative of a present state of the HCPs with respect to the given medical product at the present time point.

In some embodiments, the first attribute data can comprise one or more attributes from a set of attributes characterizing the HCPs in the first list, the set including (but not limited to): specialty, geography, historical number of patients, historical number of prescriptions, acquisition rate of new patients, tendency to switch between medical products, patient attributes (such as, e.g., patients' medical records including ages, insurance plans, line of therapy, etc, which can be sorted and aggregated to aggregated attributes), and historical events directed thereto (such as, e.g, marketing activities with respect to the given product, including campaigns/promotions directed to the HCPs).

The first attribute data can be represented in various data forms, such as, e.g., a table, a list, vectors, etc. By way of example, the first attribute data can be represented in the form of a table, where the columns contain the one or more attributes as selected from the set of attributes, and each row corresponds to a respective HCP in the first list of HCPs and contains the attribute values of the one or more attributes for the respective HCP.

It is to be noted that the present time point refers to a time point when the prediction is requested and performed in the inference phase. This term is used to differentiate from a given training time period which is prior to the present time point, as will be described below. The first list of HCPs and the first attribute data thereof as obtained pertain to the present time point. By way of example, upon receiving a request for a prediction for a given transition at a present time point, the first list of HCPs that are associated with the first stage at that specific time point is retrieved, as well as the first attribute data associated therewith. These data are used as the input fed to the ML model to be processed, thereby providing a prediction result.

A prediction is performed (206) (e.g., by the prediction module 108 of system 100) on the first list of HCPs with respect to the given transition using a machine learning (ML) model based on the first attribute data, giving rise to a second list of HCPs each associated with a respective likelihood of changing states corresponding to the given transition at the present time point. In some embodiments, the second list of HCPs is usable for prioritizing HCPs for an event/activity directed to the given transition. By way of example, the top HCPs in the second list can be selected and a specific event (such as, e.g., a marketing campaign or promotion) with respect to the given transition can be planned/tailored specifically for the selected HCPs.

Continuing with the example of FIG. 6, assume a given transition from a first stage of a continuous prescriber 606 to a second stage of a churned prescriber 608 is selected, and a prediction is requested to be performed for the selected transition at a present time point. A first list of HCPs that are associated with the first stage of a continuous prescriber 606 at the present time point are obtained, as well as the first attribute data characterizing the first list of HCPs at the present time point. The HCPs in the first list are the HCPs that are regarded as continuous prescribers of a given medical product at the present time point.

A ML model that is previously trained with respect to the selected transition (the training is detailed below with respect to FIG. 3) can be used for performing the prediction based on the first list of HCPs and the attribute data characterizing the HCPs.

The output of the ML model is a second list of HCPs each associated with a respective likelihood of the HCP changing states from a continuous prescriber to a churned prescriber at the present time point. By way of example, the second list of HCPs can contain the same HCPs as of in the first list, but the HCPs are arranged with a different ranking in the second list according to the predicted likelihood of churning. By way of another example, the second list of HCPs can contain a predefined number of HCPs from the first list that have relatively high likelihood of churning (or retain the present state of a continuous prescriber).

As described above, the ML model can be implemented as various types of machine learning models, such as, e.g., decision tree, Support Vector Machine (SVM), Artificial Neural Network (ANN), Bayesian network, ensembles/combinations thereof, or in some cases as regression models, etc, and the present disclosure is not limited to any specific implementation of the ML model.

In one embodiment, the ML model can be implemented as a Random forest model. Random forest is a supervised learning algorithm. The “forest” refers to an ensemble of decision trees. Random forest generally builds multiple decision trees and merges them together in order to get a more accurate and stable prediction. By using random forest, it adds additional randomness to the model, while growing the trees. Instead of searching for the most important feature/attribute while splitting a node, it searches for the best feature among a random subset of features. This results in a wide diversity that generally results in a better model. Another advantage is that it is relatively easy to measure the relative importance of each feature/attribute on the prediction. This can be done, e.g., by checking how much the tree nodes that use that feature reduce impurity across all trees in the forest. The feature importance can be used to decide which features/attributes to possibly drop (because they do not contribute enough) and which ones to emphasize on the other hand, thereby correcting the overfitting issues during training. As compared to other ML models/methods, random forests tend to produce highly accurate predictions in a robust manner. In addition, such models also support explainability of the prediction results (e.g., it provides information regarding the reasoning of the prediction results), and are relatively quick and efficient in the training and prediction phases.

In order for the ML model to be able to perform a prediction in the inference phase, the ML model has to be previously trained during a training phase. Turning now to FIG. 3, there is illustrated a generalized flowchart of training the ML model in accordance with certain embodiments of the presently disclosed subject matter.

Training data directed to the given transition and pertaining to a given time period can be obtained (302) (e.g., by the training module 104 of system 100). The training data can include historical attribute data characterizing a historical first list of HCPs associated with the first stage at the beginning of the given time period, and ground truth data indicative of a result of change of state for each HCP in the historical first list at the end of the given time period.

By way of example, the given time period can be a time period of the last three months preceding the present time point. The training data can include the historical first list of HCPs that are associated with the first stage at the beginning of the three months, i.e., at the time point of three months ago, and the historical attribute data characterizing the HCPs at that time, as well as the ground truth data whether each HCP in the list changed state at the end of the three months, i.e., at the present time point.

The training data can be provided in a similar data representation as the first attribute data as described above. By way of example, the training data can be represented in the form of a table, where the columns contain the one or more attributes and the ground truth of change of state, and each row corresponds to a respective HCP in the first historical list of HCPs and contains the attribute values of the one or more attributes for the respective HCP, as well as the ground truth reflecting whether the respective HCP changed state at the end of the time period (e.g., a binary result in terms of yes or no).

The training data can be fed to an initialized ML model (such as the random forest model), and the ML model can be trained (304) for the given transition (e.g., by the training module 104 of system 100) using the training data.

The ML model has a set of model parameters (such as, e.g., the weighting and/or threshold values of the nodes in the model) that are calculated and optimized during the training phase. Specifically, the initial values of the model parameters can be selected prior to training, and can be further iteratively adjusted or modified during training to achieve an optimal set of weighting and/or threshold values in a trained ML model. After each iteration, a difference can be determined between the actual output produced by the ML model and the target output associated with the respective training set of data. The difference can be referred to as an error value. Training can be determined to be complete when a cost function indicative of the error value is less than a predetermined value, or when a limited change in performance between iterations is achieved.

According to certain embodiments, the given time period can include one or more sub-periods within the given time period, and the ML model is trained using training data from each of the one or more sub-periods. For instance, a given time period can be the past year, and there may be training data available for a few sub-periods within the past year, such as the last three months, the first half of the last year, etc. In such cases, the ML model can be trained using training data from each of the sub-periods of the past year. For example, the ML model can be trained using the training data pertaining to the last three months as described above. In addition, the ML model can be trained using the training data pertaining to the first half year of the past year, which include the historical first list of HCPs that are associated with the first stage at the beginning of the first half year, and the historical attribute data characterizing the HCPs at that time, as well as the ground truth data whether each HCP in the list changed state at the end of the first half year.

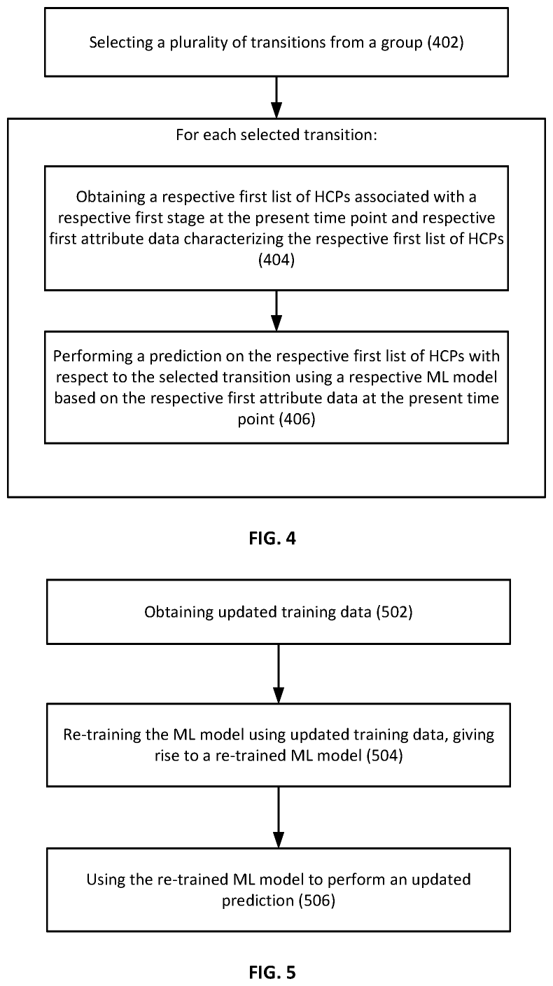

In some embodiments, the prediction process as described with reference to FIG. 2 can be performed with respect to a plurality of transitions in the HCP life cycle. Turning now to FIG. 4, there is illustrated a generalized flowchart of performing prediction for a plurality of selected transitions in accordance with certain embodiments of the presently disclosed subject matter.

A plurality of transitions can be selected (402) from the transitions in the HCP life cycle. By way of example, the plurality of transitions can be selected from a group comprising (but not limited to): a transition between not a prescriber and a new prescriber, a transition between a new prescriber and a continuous prescriber, a transition between a continuous prescriber and a churned prescriber, a transition between a churned prescriber and a new prescriber, a transition between a continuous prescriber and an increasing prescriber, and a transition between a continuous prescriber to a decreasing prescriber. As described above, the stages and/or the transitions in a HCP life cycle may be defined differently in different embodiments, therefore it is appreciated that the plurality of transitions as selected (i.e., the selected transitions) may also vary in different cases. In some embodiments, the plurality of transitions can be predetermined, e.g., by the medical/pharmaceutical organization.

For each selected transition of the plurality of selected transitions, a respective first list of HCPs associated with a respective first stage at a present time point can be obtained (404), together with respective first attribute data characterizing the respective first list of HCPs, and a prediction can be performed (406) on the respective first list of HCPs with respect to the selected transition, using a respective ML model based on the respective first attribute data at the present time point. In some embodiments, the respective ML model for each selected transition can be specifically selected according to the selected transition. By way of example, different types and/or configurations of ML models can be used for different selected transitions based on specific characteristics thereof, such as, e.g., the total number of HCPs associated with the beginning stage (i.e., the first stage) of a selected transition, the proportion of HCPs that actually undergoes the selected transition in the training data, the available data attributes characterizing the HCPs, and/or the available length of time period of the training data, etc. For instance, a DNN may be more suitable for transitions with a larger number of HCPs and/or a larger proportion of HCPs that actually undergoes the given transition in the training data, while a decision tree may be used for transitions with a smaller number/proportion of HCPs. In some cases a ML model can be specifically selected for a selected transition based on one or more of the above characteristics of the given transition.

Once the prediction is performed for all of the plurality of selected transitions, a plurality of second lists of HCPs can be generated corresponding to the plurality of selected transitions at the present time point.

In the example of FIG. 6, for instance, in addition to the transition from a first stage of a continuous prescriber 606 to a second stage of a churned prescriber 608 as described above, an additional transition from a first stage of a new prescriber 604 to a second stage of a continuous prescriber 606 can be selected. A ML model can be designed and trained for the additional transition using specific training data pertaining to the additional transition, and the trained ML model can be used in inference for performing prediction with respect to such transition.

Turning now to FIG. 5, there is illustrated a generalized flowchart of a runtime retraining process of the ML model based on updated training data in accordance with certain embodiments of the presently disclosed subject matter.

In some cases, the historical training data that is available at the training phase for a given transition can be limited due to various factors. In such cases, the ML model can be initially trained in the training phase using the available training data. In runtime, upon receiving (502) updated data pertaining to the given transition, the updated data can be used as additional training data to retrain (504) the ML model. This can be especially useful when the up-to-date data is only available on-site (i.e., at a production environment) while the ML model is initially trained in a development environment where the amount of training data is limited and not up-to-date.

Additionally or alternatively, in some embodiments, upon reviewing the prediction result (e.g, the second list of HCPs), a user (and/or a domain expert) can provide feedback with respect to the prediction result, and the ML model can be re-trained based on the user feedback. The re-trained ML model can be used (506) to generate an updated prediction result.

It is to be noted that the examples referred to herein, such as, e.g., the HCP life cycle, the stages and/or the transitions thereof, and the ML models etc. are described herein for illustrative and exemplified purposes, and should not be regarded as limiting the present disclosure in any way. Other suitable alternatives can be used in addition to, or in lieu of the above.

Among advantages of certain embodiments of the prediction process as described herein is automation of the selection/prioritization of HCPs with respect to their respective states in relation to the medical product and with respect to different transitions in the HCP life cycle. Such automation is not only computationally efficient, but also results in a better prediction result with higher accuracy and lower error rate. This is enabled at least by collecting and organizing training data specific for given transitions in the HCP life cycle, and using ML models specifically configured and trained with respect to the given transitions using such specific training data to perform predictions. This automation can enable improved planning and resource allocations for a medical/pharmaceutical organization, which in turn can increase revenues/sales of the medical products of the organization. Using a specifically selected ML model can also lead to prediction with higher accuracy and robustness.

The computerized prediction system implemented as such has an improved internal functionality with respect to, by way of example, reducing processing time of the processor for performing the prediction by utilizing specific ML models trained in specific manners, and/or parallel processing of predictions for different transitions, etc.

It is to be understood that the present disclosure is not limited in its application to the details set forth in the description contained herein or illustrated in the drawings.

It will also be understood that the system according to the present disclosure may be, at least partly, implemented on a suitably programmed computer. Likewise, the present disclosure contemplates a computer program being readable by a computer for executing the method of the present disclosure. The present disclosure further contemplates a non-transitory computer-readable memory tangibly embodying a program of instructions executable by the computer for executing the method of the present disclosure.

The present disclosure is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

Those skilled in the art will readily appreciate that various modifications and changes can be applied to the embodiments of the present disclosure as hereinbefore described without departing from its scope, defined in and by the appended claims.

1. A computerized prediction method, the computerized method performed by a processing and memory circuitry (PMC), the computerized method comprising:
obtaining a predefined health care provider (HCP) life cycle comprising: a plurality of stages indicative of a plurality of states of an HCP with respect to a given medical product, and one or more transitions each between a pair of stages of the plurality of stages and indicative of a change of state of an HCP between two states corresponding to the pair of stages;
for at least one given transition from a first stage to a second stage in the HCP life cycle, obtaining a first list of HCPs associated with the first stage indicative of a present state of the HCPs with respect to the given medical product at a present time point, and first attribute data characterizing the first list of HCPs at the present time point; and
performing a prediction on the first list of HCPs with respect to the given transition using a machine learning (ML) model based on the first attribute data, giving rise to a second list of HCPs each associated with a respective likelihood of changing states corresponding to the given transition at the present time point;
wherein the ML model is previously trained with respect to the given transition using training data pertaining to a given time period, the training data including historical attribute data characterizing a historical first list of HCPs associated with the first stage at the beginning of the given time period, and ground truth data indicative of a result of change of state for each HCP in the historical first list at the end of the given time period.

2. The computerized method according to claim 1, wherein the plurality of stages comprises at least two of the following stages: not a prescriber, a new prescriber, a continuous prescriber, an increasing prescriber, a decreasing prescriber, and a churned prescriber.

3. The computerized method according to claim 1, wherein the ML model is selected from a group comprising: decision tree, Support Vector Machine (SVM), Artificial Neural Network (ANN), Bayesian network, and an ensemble thereof.

4. The computerized method according to claim 1, wherein the first attribute data comprises one or more attributes from a set of attributes characterizing the HCPs in the first list including: specialty, geography, historical number of patients, historical number of prescriptions, acquisition rate of new patients, tendency to switch between medical products, patient attributes, and historical events directed to the HCPs.

5. The computerized method according to claim 1, wherein the given time period includes one or more sub-periods within the given time period, and wherein the ML model is trained using training data from each of the one or more sub-periods.

6. The computerized method according to claim 1, wherein the at least one given transition comprises a plurality of selected transitions, and the method comprises, for each selected transition, obtaining a respective first list of HCPs associated with a respective first stage at the present time point and respective first attribute data characterizing the respective first list of HCPs, and performing a prediction on the respective first list of HCPs with respect to the selected transition using a respective ML model based on the respective first attribute data at the present time point, giving rise to a plurality of second lists of HCPs corresponding to the plurality of selected transitions at the present time point.

7. The computerized method according to claim 1, wherein the plurality of selected transitions are selected from a group comprising: a transition between not a prescriber and a new prescriber, a transition between a new prescriber and a continuous prescriber, a transition between a continuous prescriber and a churned prescriber, a transition between a churned prescriber and a new prescriber, a transition between a continuous prescriber and an increasing prescriber, and a transition between a continuous prescriber to a decreasing prescriber.

8. The computerized method according to claim 6, wherein the respective ML model is specifically selected according to one or more characteristics of the selected transition.

9. The computerized method according to claim 1, wherein the given medical product is selected from a group comprising: a given medicine, a given medical service, a given medical device, or a given brand of medicines.

10. The computerized method according to claim 1, wherein the second list of HCPs is usable for prioritizing HCPs for an event directed to the given transition.

11. A computerized prediction system, the system comprising a processing and memory circuitry (PMC) configured to:
obtain a predefined health care provider (HCP) life cycle comprising: a plurality of stages indicative of a plurality of states of an HCP with respect to a given medical product, and one or more transitions each between a pair of stages of the plurality of stages and indicative of a change of state of an HCP between two states corresponding to the pair of stages;
for at least one given transition from a first stage to a second stage in the HCP life cycle, obtain a first list of HCPs associated with the first stage indicative of a present state of the HCPs with respect to the given medical product at a present time point, and first attribute data characterizing the first list of HCPs at the present time point; and
perform a prediction on the first list of HCPs with respect to the given transition using a machine learning (ML) model based on the first attribute data, giving rise to a second list of HCPs each associated with a respective likelihood of changing states corresponding to the given transition at the present time point;
wherein the ML model is previously trained with respect to the given transition using training data pertaining to a given time period, the training data including historical attribute data characterizing a historical first list of HCPs associated with the first stage at the beginning of the given time period, and ground truth data indicative of a result of change of state for each HCP in the historical first list at the end of the given time period.

12. The computerized system according to claim 11, wherein the plurality of stages comprises at least two of the following stages: not a prescriber, a new prescriber, a continuous prescriber, an increasing prescriber, a decreasing prescriber, and a churned prescriber.

13. The computerized system according to claim 11, wherein the first attribute data comprises one or more attributes from a set of attributes characterizing the HCPs in the first list including: specialty, geography, historical number of patients, historical number of prescriptions, acquisition rate of new patients, tendency to switch between medical products, patient attributes, and historical events directed to the HCPs.

14. The computerized system according to claim 11, wherein the given time period includes one or more sub-periods within the given time period, and wherein the ML model is trained using training data from each of the one or more sub-periods.

15. The computerized system according to claim 11, wherein the at least one given transition comprises a plurality of selected transitions, and the PMC is configured to, for each selected transition, obtain a respective first list of HCPs associated with a respective first stage at the present time point and respective first attribute data characterizing the respective first list of HCPs, and perform a prediction on the respective first list of HCPs with respect to the selected transition using a respective ML model based on the respective first attribute data at the present time point, giving rise to a plurality of second lists of HCPs corresponding to the plurality of selected transitions at the present time point.

16. The computerized system according to claim 11, wherein the plurality of selected transitions are selected from a group comprising: a transition between not a prescriber and a new prescriber, a transition between a new prescriber and a continuous prescriber, a transition between a continuous prescriber and a churned prescriber, a transition between a churned prescriber and a new prescriber, a transition between a continuous prescriber and an increasing prescriber, and a transition between a continuous prescriber to a decreasing prescriber.

17. The computerized system according to claim 15, wherein the respective ML model is specifically selected according to one or more characteristics of the selected transition.

18. The computerized system according to claim 11, wherein the given medical product is selected from a group comprising: a given medicine, a given medical service, a given medical device, or a given brand of medicines.

19. The computerized system according to claim 11, wherein the second list of HCPs is usable for prioritizing HCPs for an event directed to the given transition.

20. A non-transitory computer readable storage medium tangibly embodying a program of instructions that, when executed by a computer, cause the computer to perform a prediction method, the method comprising:
obtaining a predefined health care provider (HCP) life cycle comprising: a plurality of stages indicative of a plurality of states of an HCP with respect to a given medical product, and one or more transitions each between a pair of stages of the plurality of stages and indicative of a change of state of an HCP between two states corresponding to the pair of stages;
for at least one given transition from a first stage to a second stage in the HCP life cycle, obtaining a first list of HCPs associated with the first stage indicative of a present state of the HCPs with respect to the given medical product at a present time point, and first attribute data characterizing the first list of HCPs at the present time point; and
performing a prediction on the first list of HCPs with respect to the given transition using a machine learning (ML) model based on the first attribute data, giving rise to a second list of HCPs each associated with a respective likelihood of changing states corresponding to the given transition at the present time point;
wherein the ML model is previously trained with respect to the given transition using training data pertaining to a given time period, the training data including historical attribute data characterizing a historical first list of HCPs associated with the first stage at the beginning of the given time period, and ground truth data indicative of a result of change of state for each HCP in the historical first list at the end of the given time period.
